(19) United States
(12) Patent Application Publication
(10) Pub. No.: US 2006/0281997 A1
(43) Pub. Date: Dec. 14, 2006

(54) Arrhythmogenic risk management tools and methods of use

(76) Inventor: Juan R. Guerrero, Hannover (DE)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/447,310

(22) Filed: Jun. 6, 2006

Related U.S. Application Data
(60) Provisional application No. 60/687,335, filed on Jun. 6, 2005.

(51) Int. Cl. A61B 5/04

(52) U.S. Cl. 600/509

(57) ABSTRACT

Novel approaches to identifying and managing cardiac arrhymogenic risks associated with prolongation and/or dispersion of ventricular repolarization, torsadogenic risks and QT prolongation risks.

This application claims the benefit of priority based on U.S. Provisional Patent Application No. 60/687,335, filed Jun. 6, 2005.

The present invention relates to a novel approach to identifying and managing cardiac arrhythmogenic risks associated with prolongation and/or dispersion of ventricular repolarization. The arrhythmogenic risks may be, for example, congenital, acquired, or drug induced, such as drug-induced Long QT Syndrome. As described herein, arrhythmogenic risks encompass ectopic beats, sustained and/or non-sustained ventricular tachycardia, e.g., Torsades de Pointes and ventricular fibrillation, and torsadogenic risks and QT prolongation risks.

Accordingly, the present invention also relates to a novel approach to identifying and managing torsadogenic risk, for example, congenital, acquired, and drug induced, including but not limited to the torsadogenic risk created by antiarrhythmic drugs and other classes of drugs.

The present invention also relates to a novel approach to identifying and managing QT prolongation risk, for example, as it relates to congenital, acquired, and drug induced risk, including but not limited to the QT prolongation risk created by CNS active compounds, antihistamines, antimicrobials, and gastrointestinal drugs, and other drug classes.

As used herein, “identifying” a certain risk means determining the existence of the risk, determining the ability to increase that risk, and/or discerning the nature of the risk. Thus, “identifying the arrhythmogenic risk of a patient” may relate to determining the existence of the risk in the first place, the possible increase of the risk in certain circumstances, or the nature of the patient's risk (for example, drug induced torsadogenic risk). Determining the nature of the risk will usually require more information than determining the existence of the risk. Also, “identifying the arrhythmogenic risk of a substance” may relate to determining whether administering the substance to a patient may create or increase an arrhythmogenic risk. That risk may vary in nature; for example, it may relate to QT prolongation and/or torsadogenic risk.

The risk of cardiac arrhythmogenic events may be made evident, for example, in genetically predisposed persons or in those who acquire the risk as part of cardiovascular pathology and/or upon administration of certain drugs or combinations thereof. See, for example, P. J. Kannankeril et al., “Genetic Susceptibility to Acquired Long QT Syndrome: Pharmacological Challenge in First-Degree Relatives,” 2 Heart Rhythm 134 (2005). Among arrhythmogenic events, a torsadogenic event can be a catastrophic cardiac arrhythmia that may relate to prolonged or abnormally dispersed ventricular repolarization; the most common form is that known as “Torsades de Pointes” or “TdP,” from the work of Francois Dessertenne. See Y. G. Yap & A. J. Camm, “Drug Induced QT Prolongation and Torsades de Pointes,” 89 Heart 1363 (2003). Among the supposed or possible causes of increased torsadogenic risks, antiarrhythmic drugs have gained particular notoriety, and that possibility has led to the identification of risk factors and withdrawal of regulatory approval of a number of antiarrhythmic drugs and of non-antiarrhythmic drugs from other drug classes. See D. M. Roden, “Drug-Induced Prolongation of the QT Interval,” 350 N. Engl. J. Med. 1013 (2004). Accordingly, methods for identifying individuals at risk for torsadogenic events and preventing their exposure to potentially arrhythmogenic drugs to avoid torsadogenic risks is of paramount importance. Such methods promise, among other possible uses, to aid the development and medical use in patients of antiarrhythmic drugs such as tedisamil, and CNS active compounds, antihistamines, antimicrobials, gastrointestinal drugs, and other classes of therapeutic agents heretofore made difficult by the risks of TdP and other arrhythmogenic events.

Given the current regulatory environment, an appropriate risk management plan is mandatory for antiarrhythmic and non-antiarrhythmic drug development. Tpe, which is the interval measured from the peak to the end of the T wave in the electrocardiogram (“ECG”), is likely to be an important biomarker of arrhythmogenic risk identification and prevention.

Furthermore, the present invention relates to the measurement of cost-effective phenotypic biomarkers for facilitating patient care and risk management. Each individual has a unique genetic make-up, potentially predisposing him or her to various medical conditions that can be identified by measuring biomarkers of the individual. In some cases, those biomarkers can be readily and easily measured, with the patient experiencing a minimum of discomfort and inconvenience. Moreover, it can be beneficial for a population at risk to be evaluated for a given risk quickly and cheaply. For example, in some embodiments, patients who are hospitalized for syncope can be monitored for arrhythmogenic risk. Similarly, in other embodiments of the present invention, the first-degree relatives of a victim of sudden death syndrome can be evaluated for arrhythmogenic risk.

The peak to the end of a T wave, or “Tpe interval,” on the surface ECG reflects the transmural dispersion of ventricular repolarization across the three layers of the ventricular wall, namely the sub-epicardial, sub-endocardial, and mid-myocardial layers, each of which layers are functionally and anatomically different. Tpe prolongation and dispersion are the two major electrophysiological events (another is EAD) leading to TdP. Also, Early After Depolarization (“EAD”) can signal arrhythmogenic risk, for example, when Tpe shows prolongation. Accordingly, EAD can be a useful biomarker of TdP or arrhythmogenic risk, alone or in combination with Tpe. Another biomarker that may be useful in some patients for identifying arrhythmogenic risk is the fractionation of QRS.

In the last 80 years, the QT interval and QTc—the heart rate related correction using one of the more than 30 formulae created for this purpose—have been the main tools used to attempt identification of arrhythmogenic risk without much success. Evidence has been accumulated that indicates Tpe is a much better marker than QT/QTc to disclose ventricular repolarization pathology conducive to identifying drug-induced TdP risk and other arrhythmogenic risks.

QT/QTc is an unreliable index because: 1) QT prolongation may or may not be present in patients who have congenital or acquired TdP; hence it is not a reliable predictor. Nor is QT prolongation directly linked to the development of Torsade events; and 2) accurate measurement of QTc is difficult due to the poor resolution of the usual 12 lead ECG where 1 mm segment on the ECG represents 40 ms. That is especially true in pathologic states associated with fast or irregular heart rates such as atrial fibrillation.

TdP: Pathophysiology: The association between torsade and a prolonged QT interval has long been known, but the mechanisms involved at the cellular and ionic levels have been made clearer in approximately the last decade. The abnormality underlying both acquired and congenital long QT syndromes is in the ionic current flow during repolarization, which affects the QT interval. Various studies support the concept that prolongation of the repolarization delays the inactivation of the ion channels responsible for the inward flow of positive depolarizing currents. This leads to a further delay in repolarization and causes early after depolarization (EAD), the triggering event for torsade. The following phases are described:

Phase 1: During initial upstroke of action potential in a normal cardiac cell, a rapid net influx of positive ions (Na+ and Ca++) occurs, which results in the depolarization of the cell membrane. This is followed by a rapid transient outward potassium current (Ito), while the influx rate of positive ions (Na+, Ca++) declines. This represents the initial part of the repolarization, or phase 1.

Phase 2 is characterized by the plateau, the distinctive feature of which is the cardiac repolarization. The positive currents flowing inward and outward become almost equal during this stage.

Phase 3 of the repolarization is mediated by activation of the delayed rectifier potassium current (IK) moving outward while the inward positive current decays. If a slow inactivation of the Ca⁺⁺ and Na⁺ currents occurs, this inward “window” current can cause single or repetitive depolarization during phases 2 and 3 (i.e., EADs). These EADs appear as pathologic U waves on a surface ECG, and, when they reach a threshold, they may trigger ventricular tachyarrhythmias.

These changes in repolarization do not occur in all myocardial cells. The deep endocardial region and midmyocardial layer (composed of M cells) of the ventricle are more prone to prolongation of repolarization and EADs because they have a less rapid delayed rectifier potassium current (IKr), while other regions might have short or normal cycles. This heterogeneity of repolarization in the myocardial cells promotes the spread of triggered activity, which is initiated by EADs by a reentrant mechanism and currently is thought to be responsible for the maintenance of torsade.

Mortality/Morbidity: Torsade is a life-threatening arrhythmia and may present as sudden cardiac death in patients with structurally normal hearts.

According to the finding of the present invention it is noted that Tpe (unlike QT) is relatively stable across the range of heart rates and could be a fair parameter which can be accurately measured using the proper techniques even in conditions such as atrial fibrillation.

Furthermore, the results of ongoing pilot analysis comparing tedisamil-induced TdP cases to matching controls demonstrate the exceptional value of Tpe prolongation and dispersion before drug administration as a risk identification and prevention biomarker. The preliminary results are most encouraging and mirror similar findings in a series of unpublished studies done by the author in medicated schizophrenic patients and matching controls. Those controls were matched with the patients for sex and age, and for concurring conditions if possible.

It is also possible to evaluate the risk of QT prolongation by measuring Tpe. For example, many drugs can be classified according to their established or potential risk for causing QT prolongation and/or Torsades de Pointes. See R. L. Woolsley, “Drugs that Prolong the QT Interval and/or Induce Torsades de Pointes,” published on-line at http://www.arizonacert.org/medical-pros/drug-lists/printable-drug-list.cfm. Evaluating the arrhythmogenic risk of drugs, such as those appearing on that website and in similar sources, by measuring Tpe is included within the scope of this invention. See also, for example, www.LongQT.com.

Examples of drug classes suspected or known to create or increase arrhythmogenic risk include but are not limited to:

cardiovascular drugs such as anti-arrhythmics, anti-anginals, antihypertensives, and heart failure drugs;

gastro-intestinal drugs such as GI stimulants, anti-nausea compounds, and anti-emetics;

CNS active drugs such as anti-psychotics, anti-depressants, anti-schizophrenics, and opiate agonists;

antihistamines;

anti-microbials such as anti-malarials, antifungals, and antibiotics.

Examples of drugs suspected or known to create or increase arrhythmogenic risk include: amiodarone, arsenic trioxide, astemizole, bepridil, chloroquine, chlorpromazine, cisapride, clarithromycin, disopyramide, dofetilide, domperidone, droperidol, erythromycin, flosequinan, grepafloxacin, halofantrine, haloperidol, ibutilide, levomethadyl, mesoridazine, methadone, mibefradil, pentamidine, pentamidine, pimozide, procainamide, quinidine, sotalol, sparfloxacin, thioridazine, terfenadine, astemizole, terodiline, droperidol, lidoflazine, sertindole, levomethadyl, and tedisamil.

For more information on drugs that may have arrhythmogenic risks, see, for example, Y. G. Yap & A. J. Camm, “Drug Induced QT Prolongation and Torsades de Pointes,” 89 Heart 1363 (2003).

Thus, the findings of the present invention may be also of high interest for regulatory agencies in the pre-approval evaluation of arrhythmogenic risks such as torsadogenic risks and QT prolongation risks. The traditional QT and QTc measurements are not as reliable as previously thought, especially for predicting torsadogenic risk. A valid, pathophysiologically correct biomarker of abnormal ventricular repolarization is very necessary. One of the problems with the QT measurement is that it includes the QRS segment which depicts ventricular depolarization and has its own arrhythmogenic risk independent of that related to ventricular repolarization abnormalities such as TdP. A compounding factor is the over-reliance in obsolete and inadequate computer algorithms for automated QT measurement without overreading by a trained cardiologist. The use of a biomarker of arrhythmogenic risk should assist the pre-approval drug development process as well as the monitoring of the safety after drugs enter the general market.

Additionally, this Tpe approach to analyzing both human and animal ECGs certainly goes beyond the drug tedisamil, and would help in evaluating the torsadogenic risk for antiarrhythmic and other drugs before they are given to man for the first time.

The Tpe approach can also analyze the QT prolongation risk for various drugs. Such drugs include CNS active compounds, antihistamines, antimicrobials, and gastro-intestinal drugs, for example. Several otherwise effective drugs have been taken off of the market because of QT prolongation. At least some of those drugs potentially could be returned to the market for some patients if an effective method for screening patients for QT prolongation risk and/or torsadogenic risk were applied.

Among other embodiments, the present invention relates to ways and means to identify inadequate-heterogeneous temporal and spatial ventricular repolarization abnormalities as a biomarker for a propensity to develop cardiac arrhythmias.

Among other embodiments, this invention pertains to ways and means to identify inadequate-heterogeneous temporal and spatial ventricular repolarization abnormalities as a biomarker for a propensity to develop drug-induced Torsades de Pointes.

Among other embodiments, the present invention relates to methods for screening patients for susceptibility for drug-induced Torsades de Pointes in which the drug is an antiarrhythmic drug. The present invention, in other embodiments, relates to methods for screening patients for susceptibility for drug-induced Torsades de Pointes in which the drug is other than an antiarrhythmic drug.

It is also possible to evaluate human and animal patients for potential QT prolongation due to the administration of certain drugs by measuring the Tpe as described herein. Accordingly, some embodiments of the present invention relate to methods for screening patients for susceptibility for QT prolongation.

Arrhythmogenic risks, including torsadogenic risks and QT prolongation risks, can be caused by the combined action of more than one factor. For example, in some cases it may be good medical practice to avoid administering a drug having a potential torsadogenic risk or a potential QT prolongation risk to a patient with a family history or prior history of either of those conditions. Similarly, concurrent administration of two drugs leading to such potential risks might not be prudent. Thus, in some embodiments, the present invention relates to evaluating a patient for arrhythmogenic risk caused by more than one factor, such as concurrent administration of more than one drug with known or potential arrhythmogenic effect.

### EXAMPLES

The following non-limiting examples illustrate the invention.

1. Improvement of Data Extraction from Current ECG Files

Conventional 12 lead electrocardiograms (ECGs) were obtained by SpaceLabs (SL) according to their standard operating procedure. These ECGs were recorded at 300 samples per second (the standard in clinical work is 125 samples per seconds), and the data were received in the rawest possible form, as close as possible to the signal originally retrieved from the patient, prior to any processing or signal manipulation. Quantization (sampling in the voltage domain) was done with an 8 bit card without pre analog/digital conversion dynamic range enhancement.

For one embodiment, 12 lead ECGs were displayed at four fold the standard temporal domain resolution (100 mm/second vs. 25 mm/second) and twice the resolution in the voltage domain (20 mm/mV vs. 10 mm/mV), which combined with the above richest original digital sampling (300 s/s vs. 125 s/s) rendered 4×2×2.4=19.2 greater resolution ECGs than those that are provided in routine clinical and in most research situations. Additionally, four fold and greater magnification in the voltage domain is possible using graphic display algorithms such as, but not limited to, Photoshop™. This display was obtained without any customization at the recording time. Although readily available, so far, to the inventor's knowledge, there is no precedent of display or analysis done using the full potential of the ECG raw signal. Hence it is not obvious to the general users that the currently available ECG files can render better and improved information on ventricular repolarization than the data obtained even at highly sophisticated research centers using conventional parameters of data display.

Enhanced display parameters of conventionally recorded ECG signals are claimed as improvements from the current state of the art. Richer time and voltage domain sampling facilitates disclosure of ventricular repolarization abnormalities. Bed side ECG recording done above (but not limited to) 300 samples per second in the time domain, quantized with 16 bit cards and higher should render even superior results.

2. Measurements Done on the ECG Signal

Manual measurement of the sub segments (J point to the peak of the T wave and peak to end of the T wave) of the ventricular repolarization are not done conventionally. If attempted at the current ECG display resolution (1 mm=40 milliseconds) such measurement would lack any precision, since the targeted segment (the peak to the end of a T wave, henceforth designated as Tpe) normally ranges below 100 milliseconds (2.5 mm in the recording display). It is worth mentioning that the line with which the ECG is commonly inscribed in paper recordings has, itself, a width equal to 8 to 10 milliseconds, hence placing the tip of manual or electronic calipers on one or the other side of such line will introduce at least 10% error. In the research literature on the matter, Tpe has been calculated using digital algorithms that render Tpe by subtracting the Q-T peak segment from the Q-T end segment where the end of the T wave is identified using the “tangent method”. In the tangent method, the computer places a line over the down slope of the T wave and traces another line at the isoelectric point; the end of the T wave is taken at the place where these two lines intersect. This is done on a low resolution, highly pre-processed and degraded (e.g. down sampled, filtered, Fast Fourier transformed, compressed signal, etc.) ECG signal which is required by the inability of past millennium algorithms to analyze data files higher than 1.4 Megabytes. The precision of automated measurement obtained with obsolete algorithms working with low resolution, highly processed and downsized data files has proven unreliable and has to be questioned and remedied.

The prevalent measure of ventricular repolarization, the QT interval “corrected” for heart rate with about 30 different formulae, has failed to disclose arrhythmogenic risk. However, since it can be readily, but imprecisely measured (manual over read is required by the FDA but not done in clinical practice) with the current algorithms, QT and QTc continue to be used. However, the arrhythmogenic signal resides in the Tpe segment which gets diluted, and lost, when measured as part of the QT or QTc intervals (usually 4 to 5 fold larger than the Tpe). The QT may not elongate and the Tpe may have increased at the expense of the JT segment of the QT. This dilution and loss of signal can be seen, for example, by comparing conventional data to the high resolution raw data, visual analysis, and computerized measurements of ECG recordings described herein taken from patients with drug-induced Torsade de Pointes and matched controls.

In some embodiments, manual measurement of the Tpe is possible with high degree of precision using methodology described herein using the pre-processed, higher-than-usual resolution, ECG files described above. In some embodiments, a graphics program (for example, but not limited to Photoshop™) is used to place fiduciary markers at visually identified Q, J, peak and end of the T wave as well as at the beginning of the P wave, when present. This is done using the Adobe Photoshop™ line tool (in the pointed arrow manner with a two pixels width). Other software applications that may be used include, but are not limited to, Corel™ and Paintshop Pro™, among others. Measurements of the segments within the line markers are done, precisely, using the Photoshop measuring tool. The intervals measured are: QQ (to derive heart rate), JQ (that represents the diastolic interval) QT end (the traditional QT interval) and Tpe (the peak to the end of a T wave) that represents repolarization of the mid-myocardial region of the ventricle; the most vulnerable zone to congenital, pathologic or drug induced heterogeneity or dispersion of ventricular repolarization which puts patients at risk for ventricular arrhythmia. The meaning and measurement of QQ, JQ, and QT end are known to skilled artisans.

While different modes of display can then be rendered, one useful mode of display is the Poincare Plot method where the Tpe (or for that matter any other value) for one beat is plotted against the same value for the same segment in consecutive beats [Tpe-(Tpe-1)] in a Y-X coordinates bi-logarithmic plot. The data obtained so far strongly suggests that the normal values are clustered in the quadrant below 100 milliseconds in the Y and X axes in the patients not at risk for ventricular arrhythmia. In the series of patients analyzed who had drug-induced Torsade de Points had Tpe values mostly exceeding 100 milliseconds on the Y and X axes bespeaking of prolongation and dispersion of the repolarization in the mid-myocardial region. The inventor proposes that

Prolongation and dispersion of Tpe, displayed for example in a Poincare Plot is a biomarker that can identify patients who are at risk for arrhythmogenic events, for example, torsadogenic events such as drug-induce ventricular arrhythmia. Evaluation of biomarkers such as Tpe also can identify the arrhythmogenic risks induced by drugs, neuroadrenergic stimulation such as that occurring during physical stress, fright, anger, and other neuroadrenergically related events amongst other possible causes such as acquired cardiovascular conditions. To construct a Poincare plot, in some embodiments one can measure at least 20 or more consecutive heart beats. That measurement, in some embodiments, can be limited to (at most) 3 non identified heart beats.

In still other embodiments, cardiac beats can be measured while applying to the patient one or more stimulation maneuvers. Such stimulation maneuvers can be chosen from, for example, neuroadrenergic, thermal, and other stimulation maneuvers, and combinations thereof.

In some embodiments, the enhanced resolution of the ECG files and visual examination of that data also allows morphologic evaluation of the T wave which corroborates the numerical findings that disclose decreased repolarization reserve and heterogeneity. For instance flattening of the top of the T wave is an abnormal configuration frequently followed by double or triple hump, biphasic (+− or −+ varieties) T waves, etcetera.

Accordingly, in some embodiments, it is beneficial to amplify the voltage domain of the ECG. That is because, in the typical ECG, cardiac depolarization and contraction generate changes in the ECG signal on the order of millivolts. In contrast, repolarization generates changes in the ECG on the order of microvolts. Conventionally, the small changes due to repolarization have been ignored, or at least recorded without adequate resolution to accurately measure them.

In some embodiments, the time domain can be magnified to further illustrate the repolarization features on the ECG. This can be useful, because the Tpe can be on the order of about 100 milliseconds. Conventionally, ECGs are recorded with 1 mm=40 ms, allotting only about 2.5 mm to the Tpe.

In some embodiments of the present invention, the ECG signal can be processed, for example, to enhance the signal-to-noise ratio. In other embodiments, the first derivative (dV/dt) can be obtained. It has been observed that in the first derivative, the fastest feature often is QRS, and the second fastest feature may represent Tpe. The noise in the first derivative is often considerably slower than Tpe. Magnifying the data, for example in the voltage domain, may make these features more visible.

In some embodiments of the present invention, once arrhythmogenic risk has been identified in a patient, that risk can be assumed to be present for life and can and should be managed to prevent catastrophic arrhythmic events. Among the many ways to manage that risk, in some embodiments, medical treatment of the patient can be initiated or altered. For example, the treating physician or other medical professional can reduce or eliminate the administration of the drug perceived to be increasing the arrhythmogenic risk, such as torsadogenic and/or QT prolongation risk. In some embodiments, the patient can be administered a different drug that does not increase such risks, and/or an auxiliary medicine that combats that risk. In some embodiments, the arrhythmogenic risk identified by the methods disclosed herein can be managed by counseling and/or treating the patient for stress reduction, altering diet, increasing exercise, modifying lifestyle, and/or offering other appropriate medical advice and/or treatment known in the art.

In still other embodiments of the present invention, the arrhythmogenic risk of a substance to a human or animal patient can be identified. The arrhythmogenic risk of a substance, such as a potential new drug, can be identified by:

(a) obtaining pre-substance administration ECG data from a group of patients;

(b) administering the substance to the group of patients;

(c) obtaining post-substance administration ECG data from the group of patients;

(d) measuring at least one biomarker from the ECG data; and

(e) determining from the at least one biomarker the arrhythmogenic risk of the substance.

Optionally, a control group can be administered a placebo, and pre-placebo and post-placebo administration ECG data can be obtained from the control group. That control group can include well matched controls, such as first degree relatives (parents, siblings, children) of the patients in the group receiving the substance.

In still other embodiments of the present invention, ECG devices useful for measuring at least one biomarker that predicts arrhythmogenic risks are contemplated. Such devices, for example, can be adapted or adaptable to have a higher sampling rate, such as, for example, 300 samples per second. In some embodiments, an ECG device can have improved quantization, such as, for example, by employing a 16 bit card. In still further embodiments, an ECG device according to the invention can include software that facilitates display, measurement, and/or analysis of at least one biomarker that predicts arrhythmogenic risks. Such software optionally can have other functions, such as fully automated analysis of ECG data to yield an identification of arrhythmogenic risk for a given patient. In still further embodiments, an ECG device according to the present invention can include a corrective function, such as a defibrillator function. Automated defibrillators are currently on the market. In some embodiments, a device according to the present invention can focus on a particular biomarker such as Tpe. Further embodiments of the present invention can be constructed with the knowledge set forth in, for example, U.S. Pat. No. 6,370,423 and U.S. Patent Application Publication No. 2004/0059203 A1, published on Mar. 25, 2004. The disclosure of the foregoing patent documents are incorporated herein by reference.

Still other embodiments include adequately labeling a substance, such as a drug, that creates or increases an arrhythmogenic risk in a human or animal patient. The labeling should adequately inform health care professionals, pharmacists, and/or patients regarding the risk. That information may include identifying risk factors in patients, contraindications, and/or adverse drug interactions, and/or other relevant information. The labeling can be in any suitable form, such as a package insert, disclosure on the package itself, and literature, brochures, seminars, and websites, for example, designed to inform patients, prospective patients, family members, care givers, medical professionals, pharmacists, and/or others about the arrhythmogenic risks of using the substance. U.S. law provides that a drug shall be deemed to be misbranded unless its labeling bears such adequate warnings against use in those pathological conditions or by children where its use may be dangerous to health, or against unsafe dosage or methods or duration of administration or application, in such manner and form, as are necessary for the protection of users. See 21 U.S.C. § 352(f).

Among other embodiments, in connection with labeling, the arrhythmogenic risk can be determined according to the methods set forth above.

### BRIEF DESCRIPTION OF THE DRAWINGS

FIG. 1 is an example of a Poincare plot in a patient who did not have TdP.

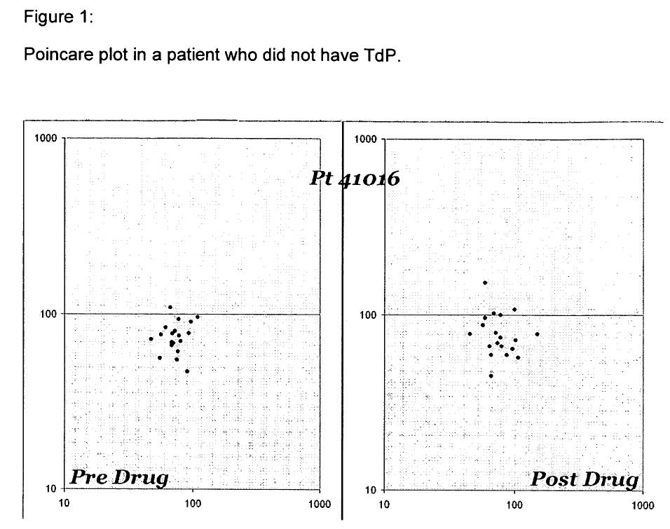

FIG. 2 is an example of a Poincare plot in a patient who had TdP.

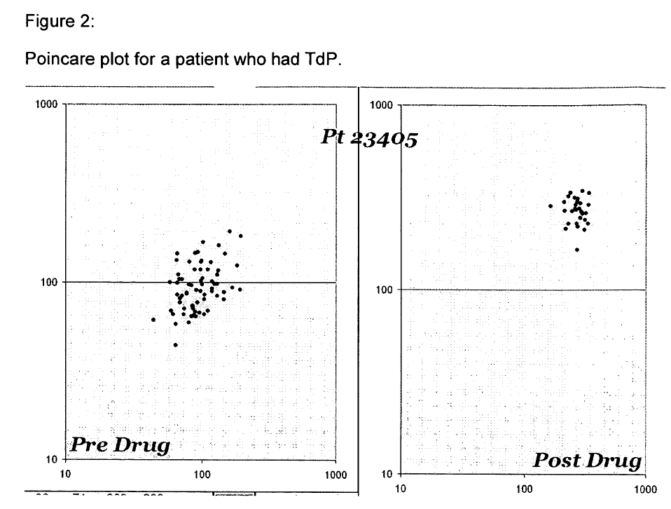

I claim:

1. A method for identifying arrhythmogenic risk in a human or animal patient, comprising:
(a) obtaining ECG data from the patient;
(b) measuring at least one biomarker from the ECG data; and
(c) determining from the at least one biomarker the arrhythmogenic risk in the patient.

2. The method of claim 1, wherein the arrhythmogenic risk is caused or increased by the administration of a drug to the patient.

3. The method of claim 1, wherein the arrhythmogenic risk is caused or increased by the administration of an antiarrhythmic drug to the patient.

4. The method of claim 3, wherein the patient is an animal.

5. The method of claim 3, wherein the patient is a human.

6. The method of claim 1, wherein the arrhythmogenic risk is caused or increased by the administration of tedisamil to the patient.

7. The method of claim 1, wherein the at least one biomarker comprises a biomarker that is the peak to the end of a T wave interval.

8. The method of claim 1, wherein the at least one biomarker is measured for more than one cardiac beat.

9. The method of claim 1, wherein the at least one biomarker for at least one cardiac beat is compared to the same biomarker for at least one preceding cardiac beat.

10. The method of claim 9, wherein the at least one cardiac beat comprises a sequence of at least 20 consecutive cardiac beats.

11. The method of claim 9, wherein the at least one cardiac beat is measured while applying to the patient at least one stimulation maneuver.

12. The method of claim 11, wherein the at least one stimulation maneuver is chosen from neuroadrenergic maneuvers, thermal maneuvers, and combinations thereof.

13. The method of claim 1, wherein the ECG data is obtained with an enhanced sampling rate.

14. The method of claim 1, wherein the ECG data is obtained with improved quantization.

15. The method of claim 13, wherein the enhanced sampling rate is greater than 125 samples per second.

16. The method of claim 13, wherein the enhanced sampling rate is equal to or greater than 300 samples per second.

17. The method of claim 1, wherein the ECG data is obtained with at least 12 leads.

18. The method of claim 1, wherein the measuring of the at least one biomarker comprises displaying the ECG data with greater than 25 mm/second temporal domain resolution, and greater than 10 mm/mV signal resolution.

19. The method of claim 1, wherein the measuring of the at least one biomarker comprises displaying the ECG data with
at least 100 mm/second temporal domain resolution, and
at least 20 mm/mV voltage domain signal resolution.

20. The method of claim 1, wherein the ECG data is obtained with greater than an 8-bit card.

21. The method of claim 1, wherein the ECG data is obtained with greater than a 12-bit card.

22. The method of claim 1, wherein the ECG data is obtained with a 16-bit or greater card.

23. The method of claim 1, wherein the measuring at least one biomarker comprises:
displaying the ECG data to yield displayed ECG data;
selecting a T wave;
marking on the displayed ECG data the peak of the T wave;
marking on the displayed ECG data the end of the T wave; and
measuring the time interval between the peak of the T wave and the end of the T wave.

24. The method of claim 23, wherein the first visualized T wave is selected.

25. The method of claim 1, wherein the measuring of the at least one biomarker comprises:
displaying the ECG data to yield displayed ECG data;
marking on the displayed ECG data and measuring one or more of the QQ interval;
the JQ interval; and
the QT interval.

26. The method of claim 1, wherein the determining from the biomarker the arrhythmogenic risk of the patient comprises plotting the biomarker of at least one cardiac beat against the same biomarker for at least one preceding cardiac beat in a bi-logarithmic plot.

27. The method of claim 1, wherein the determining from the at least one biomarker the arrhythmogenic risk of the patient comprises plotting the at least one biomarker of at least one cardiac beat against the same biomarker for at least one preceding cardiac beat in a Poincare plot.

28. The method of claim 1, wherein the at least one biomarker comprises a biomarker that is the peak to the end of a T wave interval, and the determining from the at least one biomarker the arrhythmogenic risk of the patient comprises comparing the peak to the end of a T wave interval to 100 milliseconds.

29. A method for managing arrhythmogenic risk in a human or animal patient, comprising:
(a) obtaining ECG data from the patient;
(b) measuring at least one biomarker from the ECG data;
(c) determining from the at least one biomarker the arrhythmogenic risk of the patient; and
(d) initiating or altering medical treatment of the patient to manage the determined arrhythmogenic risk of the patient.

30. The method of claim 1, wherein the arrhythmogenic risk is caused or increased by the administration of more than one drug to the patient.

31. A method for identifying QT prolongation risk in a human or animal patient, comprising:
(a) obtaining ECG data from the patient;
(b) measuring at least one biomarker from the ECG data; and
(c) determining from the at least one biomarker the QT prolongation risk of the patient.

32. The method of claim 31, wherein the QT prolongation risk is caused or increased by the administration of at least one drug to the patient.

33. The method of claim 31, wherein the QT prolongation risk is caused or increased by the administration to the patient of at least one drug chosen from CNS active compounds, antihistamines, antimicrobials, and gastro-intestinal drugs.

34. The method of claim 31, wherein the patient is an animal.

35. The method of claim 31, wherein the patient is a human.

36. The method of claim 31, wherein the QT prolongation risk is caused or increased by the administration of more than one drug to the patient.

37. The method of claim 31, wherein the at least one biomarker comprises a biomarker that is the peak to the end of a T wave interval.

38. A method for managing QT prolongation risk in a human or animal patient, comprising:
(a) obtaining ECG data from the patient;
(b) measuring at least one biomarker from the ECG data;
(c) determining from the at least one biomarker the QT prolongation risk of the patient; and
(d) initiating or altering medical treatment of the patient to manage the determined QT prolongation risk of the patient.

39. The method of claim 38, wherein the QT prolongation risk is due to the administration to the patient of at least one drug.

40. The method of claim 38, wherein the QT prolongation risk is due to the administration to the patient of at least two drugs.

41. A method for identifying torsadogenic risk in a human or animal patient, comprising:
(a) obtaining ECG data from the patient;
(b) measuring at least one biomarker from the ECG data; and
(c) determining from the at least one biomarker the torsadogenic risk of the patient.

42. The method of claim 41, wherein the torsadogenic risk is caused or increased by the administration of at least one drug to the patient.

43. The method of claim 41, wherein the torsadogenic risk is caused or increased by the administration to the patient of at least one drug chosen from anti-arrhythmics.

44. The method of claim 41, wherein the patient is an animal.

45. The method of claim 41, wherein the patient is a human.

46. The method of claim 41, wherein the torsadogenic risk is caused or increased by the administration of more than one drug to the patient.

47. The method of claim 41, wherein the at least one biomarker comprises a biomarker that is the peak to the end of a T wave interval.

48. A method for managing torsadogenic risk in a human or animal patient, comprising:
(a) obtaining ECG data from the patient;
(b) measuring at least one biomarker from the ECG data;
(c) determining from the at least one biomarker the torsadogenic risk of the patient; and
(d) initiating or altering medical treatment of the patient to manage the determined torsadogenic risk of the patient.

49. A method for identifying arrhythmogenic risk in a human or animal patient, comprising:
(a) obtaining ECG data from the patient;
(b) measuring at least one biomarker from the ECG data; and
(c) determining from the at least one biomarker the arrhythmogenic risk of the patient;
wherein the arrhythmogenic risk relates to the prolongation, dispersion, or prolongation and dispersion of ventricular repolarization.

50. A method for identifying arrhythmogenic risk in a human or animal patient, comprising:
(a) obtaining ECG data from the patient;
(b) measuring at least one Tpe from the ECG data; and
(c) determining from the at least one Tpe the arrhythmogenic risk of the patient.

51. A method for identifying arrhythmogenic risk in a human or animal patient, comprising:
(a) obtaining ECG data from the patient;
(b) displaying the ECG data with a software application;
(c) measuring at least one biomarker from the ECG data displayed with the software application; and
(d) determining from the at least one biomarker the arrhythmogenic risk of the patient.

52. The method of claim 51, wherein the software application is chosen from Corel™, Paintshop Pro™, and Adobe Photoshop™.

53. A method for identifying arrhythmogenic risk in a human or animal patient, comprising:
(a) obtaining ECG data from the patient;
(b) measuring at least one biomarker from the ECG data;
(c) plotting the at least one biomarker in a Poincare plot; and
(d) determining from the at least one biomarker plotted in the Poincare plot the arrhythmogenic risk of the patient.

54. A method for identifying arrhythmogenic risk in a human or animal patient, comprising:
(a) obtaining ECG data from the patient;
(b) displaying the ECG data with amplified voltage domain;
(c) measuring at least one biomarker from the ECG data; and
(d) determining from the at least one biomarker the arrhythmogenic risk of the patient.

55. A method for identifying the arrhythmogenic risk of a substance, comprising
(a) obtaining pre-substance administration ECG data from a group of patients;
(b) administering the substance to the group of patients;
(c) obtaining post-substance administration ECG data from the group of patients;
(d) measuring at least one biomarker from the ECG data;
(e) determining from the at least one biomarker the arrhythmogenic risk of the substance.

56. The method of claim 55, further comprising:
obtaining pre-placebo administration ECG data from a control group of patients;
administering a placebo to the control group of patients;
obtaining post-placebo administration ECG data from the control group of patients.

57. The method of claim 56, wherein the control group of patients comprises well matched controls.

58. An ECG device adapted to measure at least one biomarker of arrhythmogenic risk.

59. A method of labeling a substance that creates or increases an arrhythmogenic risk in a human or animal user of the substance, comprising:
including with the substance
adequate warnings against use in those pathological conditions or by children where its use may be dangerous to health, or against unsafe dosage or methods or duration of administration or application, in such manner and form, as are necessary for the protection of users.

60. The method of claim 59, wherein the creation or increase of arrhythmogenic risk of the substance is determined by
(a) obtaining pre-substance administration ECG data from a group of patients;
(b) administering the substance to the group of patients;
(c) obtaining post-substance administration ECG data from the group of patients;
(d) measuring at least one biomarker from the ECG data;
(e) determining from the at least one biomarker the creation or increase of arrhythmogenic risk of the substance.

61. A method of labeling a substance for administration to a human or animal patient, comprising:
including with the substance instructions for determining the arrhythmogenic risk of the patient.

62. The method of claim 61, wherein the determining the arrhythmogenic risk of the patient comprises:
(a) obtaining ECG data from the patient;
(b) measuring at least one biomarker from the ECG data; and
(c) determining from the at least one biomarker the arrhythmogenic risk in the patient.

63. The method of claim 1, further comprising:
after obtaining the ECG data, processing the ECG data to facilitate the measuring the at least one biomarker.

64. The method of claim 63, wherein the processing the ECG data comprises determining dV/dt from the ECG data.
